SEQUENCE LISTING (10 sequences)

SEQ ID NO: 1 - DNA, 22 bp, Artificial Sequence
  primer (ALPL_for)
actggtactcagacaacgagat

SEQ ID NO: 2 - DNA, 23 bp, Artificial Sequence
  primer (ALPL_rev)
acgtcaatgtccctgatgttatg

SEQ ID NO: 3 - DNA, 19 bp, Artificial Sequence
  primer (Osteonectin_for)
agcaccccattgacgggta

SEQ ID NO: 4 - DNA, 21 bp, Artificial Sequence
  primer (Osteonectin_rev)
ggtcacaggtctcgaaaaagc

SEQ ID NO: 5 - DNA, 19 bp, Artificial Sequence
  primer (Osteocalcin_for)
cactcctcgccctattggc

SEQ ID NO: 6 - DNA, 21 bp, Artificial Sequence
  primer (Osteocalcin_rev)
gcctgggtctcttcactacct

SEQ ID NO: 7 - DNA, 22 bp, Artificial Sequence
  primer (COL1A1_for)
aggacaagaggcatgtctggtt

SEQ ID NO: 8 - DNA, 17 bp, Artificial Sequence
  primer (COL1A1_rev)
ccctggccgccatactc

SEQ ID NO: 9 - DNA, 20 bp, Artificial Sequence
  primer (Runx2_for)
agtaggtgtcccgcctcaga

SEQ ID NO: 10 - DNA, 22 bp, Artificial Sequence
  primer (Runx2_rev)
  misc_feature (3)..(3): n is A or C or T.
  misc_feature (9)..(9): n is A or C or T.
ccngtgganaaaaggacttggt

(19) United States
(12) Patent Application Publication
(10) Pub. No.: US 2021/0277348 A1
(43) Pub. Date: Sep. 9, 2021

(54) METHOD AND APPARATUS FOR IN-VITRO TISSUE CULTIVATION

(71) Applicants: NANYANG TECHNOLOGICAL UNIVERSITY, Singapore (SG); CINTECHCO TECHNOLOGIES PTE LTD, Singapore (SG); QUINTECH LIFESCIENCES PTE LTD, Singapore (SG)

(72) Inventors: Swee Hin TEOH, Singapore (SG); Poh Hwa CHUA, Singapore (SG); Akhilandeshwari RAVICHANDRAN, Singapore (SG); Woon Shin CHONG, Singapore (SG); Feng WEN, Singapore (SG); Ammar Mansoor HASSANBHAI, Singapore (SG); Luvita SURYANI, Singapore (SG)

(21) Appl. No.: 16/477,861

(22) PCT Filed: Jan. 12, 2018
(86) PCT No.: PCT/SG2018/050017

§ 371 Date: Jul. 12, 2019

(30) Foreign Application Priority Data
Jan. 13, 2017 (SG) 10201700312Q

(51) Int. Cl. C12M 3/04; C12N 5/00; C12N 5/077; C12M 1/12; C12M 3/00; C12N 13/00

(52) U.S. Cl. CPC C12M 27/10; C12N 5/0068; C12N 5/0654; C12M 25/14; C12M 21/08; C12M 35/02; C12N 5/0062; C12N 2513/00; C12N 2529/00; C12N 2535/00; C12N 13/00

(57) ABSTRACT

A method for in-vitro tissue cultivation. The method may include providing a seeded-scaffold to a scaffold holder suspended in a medium contained in a bioreactor chamber. The method may further include rotating, via a rotation mechanism which the bioreactor chamber is coupled to, the bioreactor chamber about two orthogonal axes based on a predetermined motion cycle as a stimulation for tissue growth. The method may further include applying, via a stimulator coupled to the bioreactor chamber, at least one other stimulation for tissue growth to the seeded-scaffold. An apparatus for in-vitro tissue cultivation.

### TECHNICAL FIELD

Embodiments generally relate to a method for in-vitro tissue cultivation, and an apparatus for in-vitro tissue cultivation.

### BACKGROUND

Generation of in vitro engineered tissues of clinically relevant sizes is impeded by diffusion limited mass transport resulting in insufficient supply of nutrients to the cells. The mass transport limit by diffusion is <200 μm, and three-dimensional (3D) tissues that exceed this size limitation will demonstrate non-homogeneous cellular distribution when cultured under static conditions. By creating dynamic conditions and promoting nutrient transport within the scaffolds, bioreactors can generate tissues with higher cell numbers and homogeneous cellular distribution. Conventional bioreactor systems used for producing dynamic fluid flow to promote mass transport include perfusion bioreactors, spinner flasks and rotating wall vessel (RWV) bioreactors. Perfusion bioreactors enable direct perfusion of nutrient media through the scaffold pores. However, direct shear forces on the cells could wash them off the scaffolds. Spinner bioreactor is characterized by the use of a mechanical stirrer to enable mixing up of nutrients and oxygen within the system and prevent formation of gradients. Although, the set-up is relatively simple and low cost, there is non-homogeneous distribution of shear forces which could lead to unequal cellular growth on scaffolds. RWV bioreactors typically apply lesser shear stress to the cells and enable thorough mixing up of fluid. However, inherent disadvantage of this system is collision of constructs since these bioreactors do not offer anchorage to the scaffolds.

In addition to dynamic flow conditions, bioreactor systems have been developed to focus on a biomimetic approach where the mimicry of the physiological mechanical forces comes into play within the controlled environment of the bioreactor, especially for mechanically responsive tissues like bone and cartilage. Some of the commonly applied mechanical stimuli on cell seeded 3D scaffolds are shear stresses, tension, torsion and compression. Shear is one of the most commonly studied forces in the bioreactor likely due to the mimicry of blood flow in the human body. Tensile forces are more commonly studied for tissues like muscles, tendons, ligaments, blood vessels and cardiac tissues. Torsional forces have been used in engineering ligament tissues and inter-vertebral discs. Research studies that involved compressive forces have mainly focused on engineering bone and cartilage tissues.

Most of the above described studies have only demonstrated the cultivation of tissues based on a single stimulation. However in the human body, natural tissues developing under physiological conditions are always subjected to multiple stimulations. In particular, during human fetal development, natural tissue growth under physiological conditions is further subjected to multiple stimulations from the mother's womb. Thus, there is currently a lack of apparatus to mimic the natural multiple stimulations required for tissue development under physiological conditions. At most, a few studies are known to have explored the combination of a single mechanical stimulation and dynamic fluid flow conditions for engineering 3D tissues. However, these studies merely incorporate the use of perfusion pump as the sole contributor for creating a dynamic fluid environment for tissue culture in an existing bioreactor with mechanical stimulation. Such apparatus are no way close to mimicking the physiological conditions comprising multiple stimulations that are optimal for tissue development.

### SUMMARY

According to various embodiments, there is provided a method for in-vitro tissue cultivation. The method may include providing a seeded-scaffold to a scaffold holder suspended in a medium contained in a bioreactor chamber. The method may further include rotating, via a rotation mechanism which the bioreactor chamber is coupled to, the bioreactor chamber about two orthogonal axes based on a predetermined motion cycle as stimulation for tissue growth. The method may further include applying, via a stimulator coupled to the bioreactor chamber, at least one other stimulation for tissue growth to the seeded-scaffold.

According to various embodiments, there is provided an apparatus for in-vitro tissue cultivation. The apparatus may include a bioreactor chamber configured to contain a medium. The apparatus may further include a scaffold holder which is suspended in the bioreactor chamber and which is configured to receive a seeded-scaffold. The apparatus may further include a rotation mechanism as a stimulator for tissue growth. The bioreactor chamber may be coupled to the rotation mechanism. The rotation mechanism may be configured to rotate the bioreactor chamber about two orthogonal axes based on a predetermined motion cycle. The apparatus may further include at least one other stimulator for tissue growth. The at least one other stimulator may be configured to apply at least one other stimulation to the seeded-scaffold. The at least one other stimulator may be coupled to the bioreactor chamber.

### BRIEF DESCRIPTION OF THE DRAWINGS

For a more complete understanding of the description provided herein and the advantages thereof, reference is now made to the brief descriptions below, taken in connection with the accompanying drawings and detailed description, wherein like reference numerals represent like parts. In the drawings, figures are not necessarily to scale, emphasis instead generally being placed upon illustrating the principles of the invention. In the following description, various embodiments are described with reference to the following drawings.

FIG. 1 shows a schematic diagram of a method for in-vitro tissue cultivation according to various embodiments;

FIG. 2 shows a schematic diagram of an apparatus for in-vitro tissue cultivation according to various embodiments;

FIG. 3A shows a photograph for an apparatus for in-vitro tissue cultivation according to various embodiments;

FIG. 3B shows a schematic diagram of the apparatus of FIG. 3A according to various embodiments;

FIG. 3C shows a bioreactor chamber assembly of the apparatus of FIG. 3A according to various embodiments;

FIG. 3D shows an exploded of the bioreactor chamber assembly of FIG. 3C according to various embodiments;

FIG. 3E shows a cross-sectional view of the bioreactor chamber assembly of FIG. 3C according to various embodiments;

FIG. 3F shows an exploded of a stimulator of the bioreactor chamber assembly of FIG. 3C according to various embodiments;

FIG. 4 shows an apparatus for in-vitro tissue cultivation according to various embodiments;

FIG. 5 shows an apparatus for in-vitro tissue cultivation according to various embodiments;

FIG. 6 shows experimental data in the form of a plot depicting the maintenance of pH (left y-axis) and dissolved oxygen (O2) levels (right y-axis) over two weeks under static mode and multimodal mode;

FIG. 7 shows experimental data illustrating the expression levels of osteogenic genes—ALPL (alkaline phosphatase, tissue-nonspecific isozyme), COL1A1 (collagen, type I, apha 1), Runx2 (runt-related transcription factor 2), Osteonectin and Osteocalcin under the different modes;

FIG. 8 shows experimental data illustrating fold increment and statistical significance under different modes from 7 to 14 days;

FIG. 9 shows experimental data illustrating qualitative staining for calcium and phosphate in the tissues by the end of week 2; and

FIG. 10 shows experimental data illustrating matrix deposition in the tissue grafts under different modes.

### DETAILED DESCRIPTION

Embodiments described below in context of the apparatus are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural references unless context clearly indicates otherwise. Similarly, the word “or” is intended to include “and” unless the context clearly indicates otherwise.

Various embodiments of a method and an apparatus for in-vitro tissue cultivation have been provided to mimic natural conditions for tissue development including one or more physiological conditions or stimulations in combination with a “fetal gyroscopic motion” inside the womb of a pregnant woman. The one or more physiological conditions or stimulations may include dynamic fluid flow conditions and mechanical stimuli such as shear stress, tension, torsion and compression. The “fetal gyroscopic motion” may include spinning (i.e. rotating about the z-axis) and tumbling (i.e. rotating about the x-axis) simultaneously. Accordingly, various embodiments may provide a biaxial rotation to a bioreactor chamber having one or more physiological stimuli for in-vitro tissue cultivation.

Various embodiments have provided a method and an apparatus for in-vitro tissue cultivation, for example, a physiologic bioreactor that is configured to allow mechanical manipulation of tissue grafts cultured under biaxial rotation conditions. No known methods or apparatus have been reported that combines biaxial rotation with mechanical manipulation or other physiological conditions or stimulations. According to various embodiments, the physiological conditions or stimulations of the various methods and apparatus may include dynamic fluid flow, mechanical stimulation, magnetic stimulation, electrical stimulation, photo stimulation, or any other suitable stimulation that may promote tissue growth. According to various embodiments, the physiologic bioreactor may be integrated with dissolved oxygen and pH (potential of hydrogen) sensors (PreSens®) to allow non-invasive sensing of culture parameters.

Various embodiments of the methods and apparatus may present the first step towards development of bioreactor systems that may provide physiologically relevant culture conditions to generate clinically relevant functional tissue grafts. Further, the integration with non-invasive sensing modalities in the various embodiments may enable rapid, real-time sensing of bioreactor culture parameters. The physiologic bioreactor, according to the various methods and apparatus, may be used for in-vitro tissue cultivation under at least four different modes: static mode, single physiological condition or stimulation mode, biaxial rotation mode, or a multimodal mode.

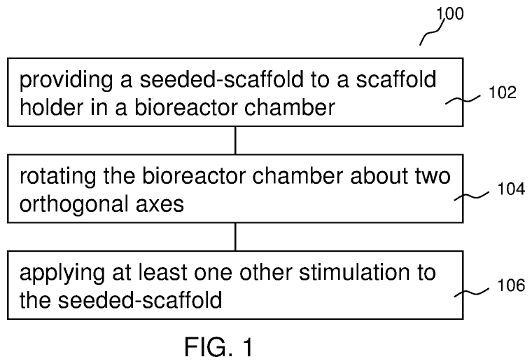

FIG. 1 shows a schematic diagram of a method 100 for in-vitro tissue cultivation according to various embodiments. The method 100 may include, at 102, providing a seeded-scaffold to a scaffold holder in a bioreactor chamber. According to various embodiments, the scaffold holder may be suspended in a medium contained in the bioreactor chamber. According to various embodiments, the bioreactor chamber may be an enclosed chamber fully filled with medium. The medium may any fluid culture medium suitable for tissue cultivation.

According to various embodiments, the method 100 may further include, at 104, rotating the bioreactor chamber about two orthogonal axes as stimulation for tissue growth. The two orthogonal axes may intersect inside the bioreactor chamber such that the bioreactor chamber may be subjected to biaxial rotation so as to generate the “fetal gyroscopic motion”. According to various embodiments, the two orthogonal axes may lay in a plane intersecting the scaffold holder such that the scaffold holder may experience the same biaxial rotation as the bioreactor chamber. According to various embodiments, the bioreactor chamber may be coupled to a rotation mechanism configured to rotate the bioreactor chamber about the two orthogonal axes. According to various embodiments, the biaxial rotation of the bioreactor chamber may be based on a predetermined motion cycle.

According to various embodiments, the method 100 may further include, at 106, applying at least one other stimulation for tissue growth (in addition to the biaxial rotation) to the seeded-scaffold on the scaffold holder. According to various embodiments, the at least one other stimulation may include at least one of a mechanical stimulation, a magnetic stimulation, an electrical stimulation, a stimulation with one or more light sources, or any other suitable stimulation that may induce tissue growth. According to various embodiments, the at least one other stimulation may be provided by a stimulator coupled directly to the bioreactor chamber.

According to various embodiments, the scaffold holder may be elongated. Accordingly, providing the seeded-scaffold in 102 may include placing the seeded-scaffold to surround the scaffold holder and retaining the seeded-scaffold on the scaffold holder with a retainer. Hence, the seeded-scaffold may be hollow along the longitudinal axis such that the seeded-scaffold may be placed over and around the scaffold holder in order for the scaffold holder to be inserted through the seeded-scaffold. Further, a retainer may be coupled to an end of the elongated scaffold holder so as to prevent the seeded-scaffold from sliding out of the scaffold holder. Thus, the retainer may be for retaining the seeded-scaffold on the elongated scaffold holder. According to various embodiments, the scaffold holder may extend from a portion of a ceiling of the bioreactor chamber to a center region of the bioreactor chamber. Accordingly, a top end (or one end) of the scaffold holder may be joined or coupled or attached to the bioreactor chamber at the ceiling of the bioreactor chamber. Hence, the retainer may be coupled to a bottom end (or another end) of the scaffold holder. According to various embodiments, the bioreactor chamber may include a main container body and a lid covering an opening of the main container body. The elongated scaffold holder may be joined or coupled or attached to the lid of the bioreactor chamber.

According to various embodiments, rotating the bioreactor chamber via the rotation mechanism at 104 may include rotating a bracket rotatably mounted to a base of the rotation mechanism, and rotating a stage rotatably mounted to the bracket. According to various embodiments, the rotational axis of the stage and the rotational axis of the bracket may be the two orthogonal axes in 104. Accordingly, the rotational axis of the stage and the rotational axis of the bracket may be orthogonal to each other. Further, the bioreactor chamber may be coupled to the stage such that the bioreactor chamber may be rotated by the rotation of the stage and the bracket of the rotation mechanism.

According to various embodiments, the predetermined motion cycle of the biaxial rotation of the bioreactor chamber may be defined by one or more parameters selected from the group consisting of an angle of rotation, a direction of rotation, a rotational speed, a sequence of rotation, a period of rotation and a time interval between rotations. The angle of rotation may be how much the bioreactor chamber is rotated, for example 45°, 90°, 135°, 180°, etc. The direction of rotation may be, for example, either clockwise or anti-clockwise. A rotational speed may be measured, for example, in terms of rounds per minute (RPM). The sequence of rotation may be, for example, whether the bioreactor chamber is rotated about the two orthogonal axes simultaneously or one after another. The period of rotation may be, for example, a continuous duration which the bioreactor chamber is subjected to rotation. The time interval between rotations may be, for example, a break duration in between cycles of rotation.

According to various embodiments, applying the at least one other stimulation to the seeded-scaffold at 106 may include applying a mechanical stimulation to the seeded-scaffold. According to various embodiments, applying the mechanical stimulation to the seeded-scaffold may include moving the scaffold holder, via an actuator of the stimulator, relative to the bioreactor chamber to apply a compression or a tension to the seeded-scaffold based on a predetermined mechanical stimulation cycle. For example, the scaffold holder may be an elongated shaft inserted through a through-hole in the lid of the bioreactor chamber. The actuator of the stimulator may be coupled to the end of the elongated scaffold holder protruding from an exterior surface of the lid of the bioreactor chamber. Accordingly, the actuator may move or slide the elongated scaffold holder through the through-hole inward and/or outward relative to the lid of the bioreactor chamber. In this manner, the seeded-scaffold retained on a portion of the scaffold holder within the bioreactor chamber may be compressed or tensioned.

According to various embodiments, the predetermined mechanical stimulation cycle may be defined by one or more parameters selected from the group consisting of an amount of relative movement, a speed of relative movement, a period of relative movement, a sequence of relative movement, a force applied, and a time interval between compression or tension. The amount of relative movement may, for example, be a relative distance to be moved between the elongated scaffold holder and the lid of the bioreactor chamber. The speed of relative movement may, for example, in terms of meter per second. The period of relative movement may, for example, be a continuous during which the scaffold holder is moving relative to the lid of the bioreactor chamber. The sequence of relative movement may, for example, be a pattern of applying compressions or tensions. The force applied may, for example, be an amount of force exerted by the actuator. The time interval between compressions or tensions may a duration between two successive compressions or tensions.

According to various embodiments, applying the at least one other stimulation to the seeded-scaffold at 106 may include applying a magnetic stimulation to the seeded-scaffold. According to various embodiments, applying the magnetic stimulation may include generating a magnetic field, via an electromagnet arrangement of the stimulator, through the bioreactor chamber based on a predetermined magnetic stimulation cycle. According to various embodiments, the electromagnet arrangement may include a set of Helmholtz coil. For example, one solenoid electromagnet of the set of Helmholtz coil may be at the lid of the bioreactor chamber (or a top of the bioreactor chamber) and one other solenoid electromagnet of the set of Helmholtz coil may be at a base of the bioreactor chamber (or a bottom of the bioreactor chamber). According to various embodiments, the electromagnet arrangement may include three sets of Helmholtz coils arranged along three orthogonal axes. Accordingly, six solenoid electromagnets may box up the bioreactor chamber such that there is a top solenoid electromagnet, a bottom solenoid electromagnet and four side solenoid electromagnets arranged in a box arrangement. Accordingly, with the six solenoid electromagnets arrangement (or the three sets of Helmholtz coils), a magnetic field through the bioreactor chamber may be generated in any direction.

According to various embodiments, the predetermined magnetic stimulation cycle may be defined by one or more parameters selected from the group consisting of a magnitude of the magnetic field, a magnetic flux, a direction of the magnetic field, a period of magnetic field generation, a sequence of magnetic field generation, and a time interval between magnetic field generations. The period of magnetic field generation may, for example, be a continuous duration whereby the electromagnet is activated to generate the magnetic field. The sequence of magnetic field generation may, for example, be an order of the direction of the magnetic field generated. The time interval between magnetic field generations may, for example, be a duration of a break in between two successive activation of the electromagnet to generate magnetic field.

According to various embodiments, applying the at least one other stimulation to the seeded-scaffold at 106 may include applying an electrical stimulation to the seeded-scaffold. According to various embodiments, applying the electrical stimulation may include generating a current, via a pair of electrodes of the stimulator, through the seeded-scaffold based on a predetermined electrical stimulation cycle. For example, the pair of electrodes may be a pair of electrode rods inserted into the bioreactor chamber through the lid of the bioreactor chamber. The pair of electrodes may be connected to an external circuit so as to generate a current passing through the medium and through the seeded-scaffold.

According to various embodiments, the predetermined electrical stimulation cycle may be defined by one or more parameters selected from the group consisting of a magnitude of the current, a direction of the current, a period of current generation, a sequence of current generation, and a time interval between current generations. The magnitude of the current may, for example, be the amount of the current. The direction of the current may, for example, be the flow direction of the current. The period of current generation may, for example, be a continuous duration whereby the current is generated. The sequence of current generation may, for example, be an order of various magnitudes and periods of current generation. The time interval between current generations may, for example, be a break duration between two successive current generations.

According to various embodiments, applying the at least one other stimulation to the seeded-scaffold at 106 may include perfusing the medium through the seeded-scaffold. According to various embodiments, perfusing the medium through the seeded-scaffold may include pumping the medium through the bioreactor chamber to perfuse the medium through the seeded-scaffold. Thus, according to various embodiments, a pump may be in fluid connection with the bioreactor chamber. At least one medium reservoir may also be in fluid connection with the pump and the bioreactor chamber. Accordingly, the pump, the at least one medium reservoir and the bioreactor chamber may be in fluid connection forming a loop such that the pump may pump the medium from the at least one medium reservoir into the bioreactor chamber for perfusing through the seeded-scaffold in the bioreactor chamber and back into the at least one medium reservoir. According to various embodiments, the medium may be perfused through the seeded-scaffold in the bioreactor chamber by pumping the medium via the pump based on a predetermined pump rate.

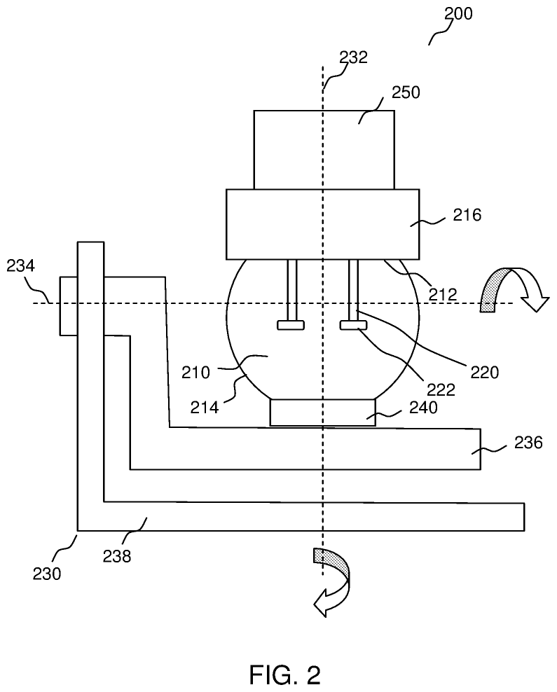

FIG. 2 shows a schematic diagram of an apparatus 200 for in-vitro tissue cultivation according to various embodiments. The apparatus 200 may include a bioreactor chamber 210 configured to contain a medium. The medium may include any fluid culture medium suitable for tissue cultivation. According to various embodiments, the bioreactor chamber 210 may be an enclosed chamber which may be fully filled with the medium. According to various embodiments, a scaffold holder 220 may be suspended in the bioreactor chamber 210. The scaffold holder 220 may be configured to receive a seeded-scaffold. According to various embodiments, the apparatus 200 may further include a rotation mechanism 230 as a stimulator for tissue growth. The bioreactor chamber 210 may be coupled to the rotation mechanism 230. Further, the rotation mechanism 230 may be configured to rotate the bioreactor chamber about two orthogonal axes 232, 234 based on a predetermined motion cycle. According to various embodiments, the apparatus 200 may further include at least one other stimulator 250 for tissue growth. The at least one other stimulator may be coupled to the bioreactor chamber 210. The at least one other stimulator 250 may be configured to apply at least one other stimulation (in addition to biaxial rotation) to the seeded-scaffold. The at least one other stimulation may include a mechanical stimulation, a magnetic stimulation, an electrical stimulation, a stimulation with one or more light sources, or any other suitable stimulation that may promote tissue growth. The at least one other stimulator 250 as shown in FIG. 2 is for illustration purposes only. According to various embodiments, the at least one other stimulator 250 may be of various shapes and configurations depending of the types of components required to provide the desired stimulation. Further, the at least one other stimulator 250 may also be coupled, either directly or indirectly, to various portions of the bioreactor chamber 210, for example, the top, the bottom and/or the sides, depending on the type of stimulator as well as the desired influence/direction of the stimulation.

According to various embodiments, the scaffold holder 220 may be elongated. Accordingly, the scaffold holder 220 may be configured to receive the seeded-scaffold such that the seeded-scaffold may surround the scaffold holder 220. Further, a retainer 222 may be coupled to the scaffold holder 220 to retain the seeded-scaffold on the scaffold holder 220. According to various embodiments, the seeded-scaffold may be in the form of a hollow tube. Accordingly, the seeded-scaffold may be placed over and around the scaffold holder 220 such that the scaffold holder 220 may be inserted through the seeded-scaffold. Further, a retainer 222 may be coupled to an end of the elongated scaffold holder 220 so as to prevent the seeded-scaffold from sliding out of the scaffold holder 220. Accordingly, the retainer 222 may be for retaining the seeded-scaffold on the elongated scaffold holder 220. According to various embodiments, the scaffold holder 220 may extend from a portion of a ceiling 212 of the bioreactor chamber 210 to a center region of the bioreactor chamber 210. Accordingly, a top end (or one end) 224 of the scaffold holder 220 may be joined or coupled or attached to the bioreactor chamber 210 at the ceiling 212 of the bioreactor chamber 220. Hence, the retainer 222 may be coupled to a bottom end (or another end) of the scaffold holder 220. According to various embodiments, the bioreactor chamber 210 may include a main container body 214 and a lid 216 covering an opening of the main container body 214. The elongated scaffold holder 220 may be joined or coupled or attached to the lid 216 of the bioreactor chamber 210.

According to various embodiments, the rotation mechanism 230 may include a bracket 236 rotatably mounted to a base 238 of the rotation mechanism. The rotation mechanism 230 may further include a stage 240 rotatably mounted to the bracket 236. According to various embodiments, the rotational axis 232 of the stage 240 and the rotational axis 234 of the bracket 236 may be orthogonal to each other. Further, the bioreactor chamber 210 may be coupled to the stage 240 such that the bioreactor chamber 210 may be rotated by the rotation of the stage 240 and the bracket 236 of the rotation mechanism 230.

According to various embodiments, the rotational mechanism 230 may be configured to define the predetermined motion cycle of the biaxial rotation of the bioreactor chamber by one or more parameters selected from the group consisting of an angle of rotation, a direction of rotation, a rotational speed, a sequence of rotation, a period of rotation and a time interval between rotations. According to various embodiments, the rotational mechanism 230 may include one or more actuators to drive the rotation of the bracket 236 relative to the base 238 and to drive the rotation of the stage 240 relative to the bracket 236. Further, the rotational mechanism 230 may include at least one controller or processor to control the one or more actuators based on the predetermined motion cycle. Furthermore, the rotational mechanism 230 may include or may be connected to a user interface for a user to input the one or more parameters defining the predetermined motion cycle.

According to various embodiments, the at least one other stimulator 250 may include a mechanical stimulator configured to apply a mechanical stimulation to the seeded-scaffold. The mechanical stimulator may include an actuator configured to move the scaffold holder 220 relative to the bioreactor chamber 210 to apply a compression or a tension to the seeded-scaffold based on a predetermined mechanical stimulation cycle for applying the mechanical stimulation. Accordingly, the scaffold holder 220 in the form of an elongated shaft may be inserted perpendicularly through a through-hole in the lid 216 of the bioreactor chamber 210. The actuator of the mechanical stimulator may be coupled to the end of the elongated scaffold holder 220 protruding from an exterior surface of the lid 216 of the bioreactor chamber 210. Accordingly, the actuator may move or slide the elongated scaffold holder 220 through the through-hole inward and/or outward relative to the lid 216 of the bioreactor chamber 210. In this manner, the seeded-scaffold retained on a portion of the scaffold holder 220 within the bioreactor chamber 210 may be compressed or tensioned accordingly.

According to various embodiments, the mechanical stimulator may be configured to define the predetermined mechanical stimulation cycle by one or more parameters selected from the group consisting of an amount of relative movement, a speed of relative movement, a period of relative movement, a sequence of relative movement, a force applied, and a time interval between compressions or tensions. Accordingly, the mechanical stimulator may include at least one controller or processor to control the actuator for moving the scaffold holder 220 based on the predetermined mechanical stimulation cycle. Further, the mechanical stimulator may include or may be connected to a user interface for a user to input the one or more parameters defining the predetermined mechanical stimulation cycle.

According to various embodiments, the at least one other stimulator 250 may include a magnetic stimulator configured to apply a magnetic stimulation to the seeded-scaffold. The magnetic stimulator may include an electromagnet arrangement configured to generate a magnetic field through the bioreactor chamber based on a predetermined magnetic stimulation cycle for applying the magnetic stimulation. According to various embodiments, the electromagnet arrangement may include one set of Helmholtz coil. The one set of Helmholtz coil may include two solenoid electromagnets arranged on a same axis. Accordingly, one solenoid electromagnet of the one set of Helmholtz coil may be disposed at the lid of the bioreactor chamber (or a top of the bioreactor chamber) and one other solenoid electromagnet of the one set of Helmholtz coil may be disposed at a base of the bioreactor chamber (or a bottom of the bioreactor chamber) such that the two solenoid electromagnets of the one set of Helmholtz coil may be in a coaxial arrangement. According to various embodiments, the electromagnet arrangement may include three sets of Helmholtz coils arranged along three orthogonal axes. Accordingly, six solenoid electromagnets may box up the bioreactor chamber 210 such that there is a top solenoid electromagnet, a bottom solenoid electromagnet and four side solenoid electromagnets arranged in a box arrangement. The top solenoid electromagnet and the bottom solenoid electromagnet may be in a coaxial arrangement. Two opposing solenoid electromagnets of the four side solenoid electromagnets may be in a coaxial arrangement. Similarly, the other two opposing solenoid electromagnets of the four side solenoid electromagnets may also be in a coaxial arrangement. Accordingly, with the six solenoid electromagnets arrangement (or the three sets of Helmholtz coils), a magnetic field through the bioreactor chamber 210 may be generated in any direction.

According to various embodiments, the magnetic stimulator may be configured to define the predetermined magnetic stimulation cycle by one or more parameters selected from the group consisting of a magnitude of the magnetic field, a magnetic flux, a direction of the magnetic field, a period of magnetic field generation, a sequence of magnetic field generation, and a time interval between magnetic field generations. Accordingly, the magnetic stimulator may include at least one controller or processor to control the electromagnet arrangement configured for generating a magnetic field through the bioreactor chamber 210 based on the predetermined magnetic stimulation cycle. Further, the magnetic stimulator may include or may be connected to a user interface for a user to input the one or more parameters defining the predetermined magnetic stimulation cycle.

According to various embodiments, the at least one other stimulator 250 may include an electrical stimulator configured to apply an electrical stimulation to the seeded-scaffold. According to various embodiments, the electrical stimulator may include a pair of electrodes configured to generate a current through the seeded-scaffold based on a predetermined electrical stimulation cycle for applying the electrical stimulation. The pair of electrodes may be a pair of electrode rods inserted into the bioreactor chamber 210 through the lid 216 of the bioreactor chamber 210. Further, the pair of electrodes may be connected to an external circuit so as to generate a current passing through the medium and through the seeded-scaffold on the scaffold holder 220.

According to various embodiments, the electrical stimulator may be configured to define the predetermined electrical stimulation cycle by one or more parameters selected from the group consisting of a magnitude of the current, a direction of the current, a period of current generation, a sequence of current generation, and a time interval between current generations. Accordingly, the electrical stimulator may include at least one controller or processor to control the pair of electrodes and/or the external circuit connected to the pair of electrodes for generating the current to pass through the seeded-scaffold on the scaffold holder 220. Further, the electrical stimulator may include or may be connected to a user interface for a user to input the one or more parameters defining the predetermined electrical stimulation cycle.

According to various embodiments, the at least one other stimulator 250 may include a perfusion flow system. The perfusion flow system may include a pump in fluid connection with the bioreactor chamber 210. The perfusion flow system may also include at least one medium reservoir in fluid connection with the bioreactor chamber 210 and the pump. Hence, the pump, the at least one medium reservoir and the bioreactor chamber 210 may be in fluid connection forming a loop such that the pump may pump the medium from the at least one medium reservoir into the bioreactor chamber 210 for perfusing through the seeded-scaffold in the bioreactor chamber 210 and back into the at least one medium reservoir. According to various embodiments, the medium may be perfused through the seeded-scaffold in the bioreactor chamber 210 by pumping the medium via the pump based on a predetermined pump rate. According to various embodiments, the perfusion flow system may also include at least one controller or processor to control the operation of the pump. Further, the perfusion flow system may include or may be connected to a user interface for a user to input the desired pump rate.

According to various embodiments, the apparatus 200 may also include a main controller or a main processor to control the operations of all the various components, including the rotation mechanism 230 and the at least one stimulator 250 (i.e. the mechanical stimulator, the magnetic stimulator, the electrical stimulator, and/or the perfusion flow system, etc.). The main controller or the main processor may be directly controlling the respective components, or may be communicating with local controllers or local processors of the respective components to control said components. Further, the main controller or the main processor may be connected to a user interface for a user to input the desired pump rate. According to various embodiments, the controller(s) or the processor(s) of the various embodiments may be understood as any kind of a logic implementing entity, which may be special purpose circuitry or processor executing software stored in a memory, firmware, or any combination thereof. Thus, the controller(s) or the processor(s) of the various embodiments may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). The controller(s) or the processor(s) of the various embodiments may also be a processor executing software, e.g. any kind of computer program, e.g. a computer program using a virtual machine code such as e.g. Java.

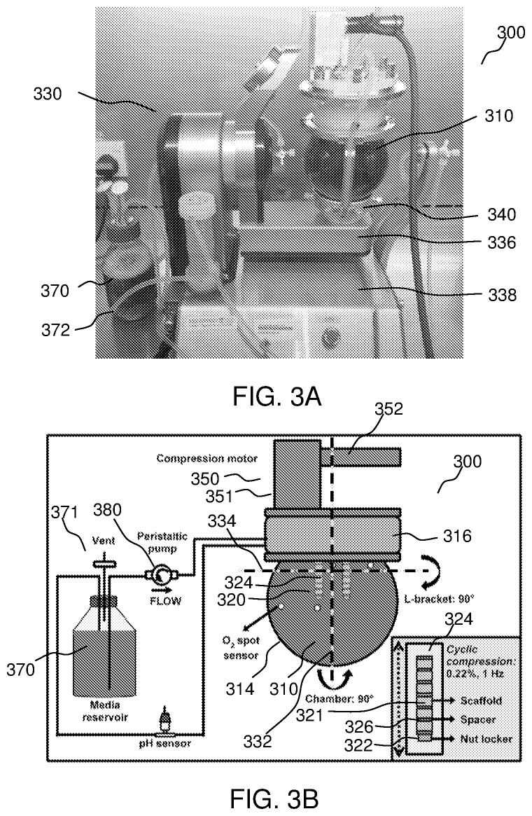

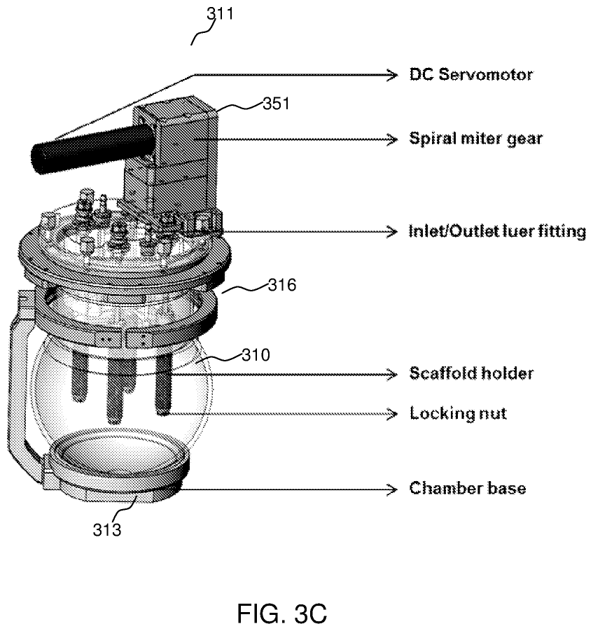

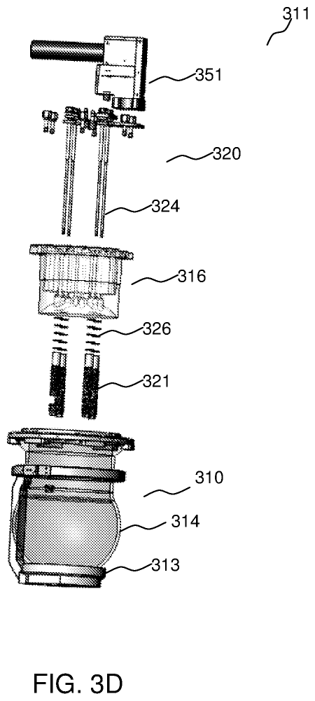

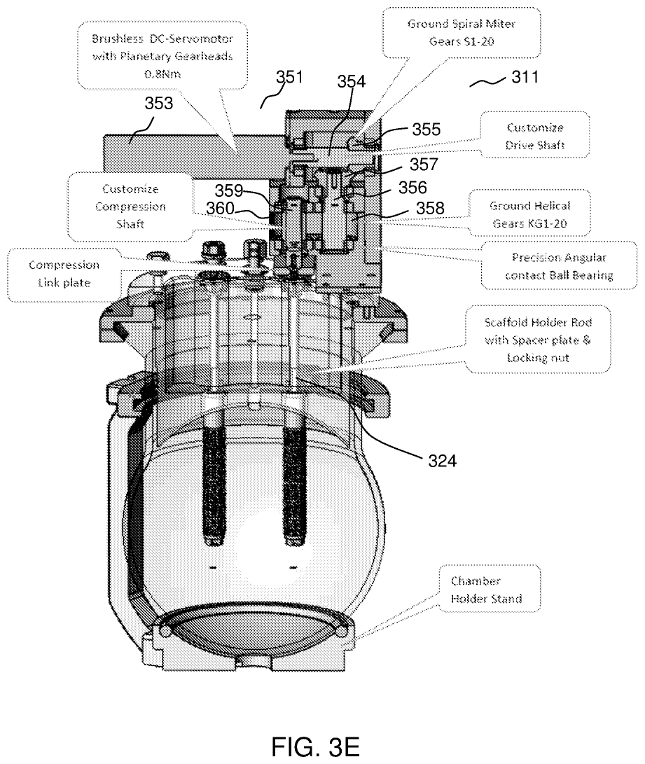

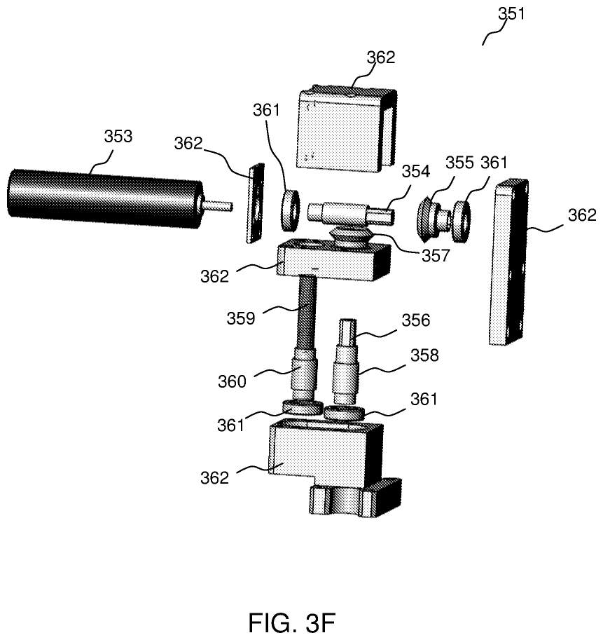

FIG. 3A shows a photograph for an apparatus 300 for in-vitro tissue cultivation according to various embodiments. FIG. 3B shows a schematic diagram of the apparatus 300 as shown in FIG. 3A according to various embodiments. FIG. 3C shows a bioreactor chamber assembly 311 of the apparatus 300 of FIG. 3A according to various embodiments. FIG. 3D shows an exploded of the bioreactor chamber assembly 311 of FIG. 3C. FIG. 3E shows a cross-sectional view of the bioreactor chamber assembly 311 of FIG. 3C. FIG. 3F shows an exploded of a stimulator 350 of the bioreactor chamber assembly 311 of FIG. 3C.

According to various embodiments, the apparatus 300 may be a biaxial rotation bioreactor having micromanipulator to apply cyclic compression on the growing tissue engineered construct. The apparatus 300 (or the bioreactor system or the physiologic bioreactor system) may include a bioreactor chamber 310 which may be a biaxial rotation chamber (with a capacity of approximately 1 liters) for containing the scaffolds therein, a media reservoir 370 (with a capacity of approximately 500 milliliters), gas-permeable tubings 372 to allow media flow between the bioreactor chamber 310 and the media reservoir 370, a peristaltic pump 380 for media perfusion, and a compression motor 352 to apply cyclic compression on scaffolds. The parameters for media perfusion, biaxial rotation and compression can be controlled by an external controller unit. The bioreactor lid 316 may include two scaffold holders 320 in the form of two shafts 324 to anchor or retain or hold or support the scaffolds and each shaft 324 may hold up to 6 scaffolds arranged in series on the shaft 324. Oxygen spot sensors (for example oxygen sensor model SP-PSt3 from PreSens®) may be adhered using silicone glue to the inner wall of the bioreactor chamber 310 and left to dry overnight. The bioreactor chamber 310 may be sterilized along with all the components that come in contact with the media using steam sterilization (for example at 121° C. for 30 min). The premature grafts may be transferred to the anchor shaft 324 in a sterile manner and separated by metallic spacers 326 (assumed incompressible). The flow-through sterile pH sensor (for example sensor model FTC-SU-HP8 from PreSens®) may be placed in the media loop at a location that allows easy access to the detector.

According to various embodiments, the apparatus 300 may be operated to perform in-vitro tissue cultivation under at least four different modes: static mode, cyclic compression mode, biaxial rotation mode, or a multimodal mode. In static mode, in-vitro tissue cultivation is maintained in the bioreactor chamber 310 with only media perfusion using the peristaltic pump 380 (for example at a speed of 5 rpm). The static mode may act as the baseline for all the modes. In the cyclic compression mode, cyclic compression may include compression at 0.22%, 1 Hz for 4 hours/day. In biaxial rotation mode, biaxial rotation may involve chamber rotation about two axes (x-axis and z-axis) at approximately 5 rpm and an angle of rotation of 90°. The final mode may be the multimodal mode which is a combination of biaxial rotation and cyclic compression at the same parameters.

Referring back to FIG. 3A and FIG. 3B, the apparatus 300 for in-vitro tissue cultivation may include the bioreactor chamber 310 configured for containing a medium or media suitable for tissue cultivation. As shown, the bioreactor chamber 310 may include a substantially spherical main container body 314. The spherical main container body 314 may include an opening or an access which is covered by the lid 316. Accordingly, the spherical main container body 314 and the lid 316 may enclose a cavity or a space to form the bioreactor chamber 310. According to various embodiments, the apparatus 300 may further include a scaffold holder 320 which may be suspended in the bioreactor chamber 310. As shown, the scaffold holder 320 may be in the form of the shaft 324. Accordingly, the scaffold holder 320 may receive a cylindrical hollow seeded-scaffold 321 such that the cylindrical hollow seeded-scaffold 321 may be put onto the scaffold holder 320 with the shaft 324 passing through the hollow channel of the cylindrical hollow seeded-scaffold 321. As shown in FIG. 3B, one or more seeded-scaffold 321 may be placed on one shaft 324. In between two adjacent seeded-scaffolds 321, a spacer element 326 may be inserted to separate the two adjacent seeded-scaffolds 321. Further, at a free end or tip of the shaft 324, a retainer 322 (for example a locking nut) may be attached such that the seeded-scaffolds 321 and the spacers 326 may be prevented from sliding out of the shaft 324. Hence, the retainer 322 may retain or hold or maintain or keep the seeded-scaffolds 321 and the spacers 326 on the shaft 324.

As shown in FIG. 3A, the apparatus 300 may further include a rotation mechanism 330 as a stimulator for tissue growth. The bioreactor chamber 310 may be coupled to the rotation mechanism 330. Further, the rotation mechanism 230 may be configured to rotate the bioreactor chamber about two orthogonal axes 332, 334 based on a predetermined motion cycle. As shown, the rotation mechanism 330 may include a bracket 336 rotatably mounted to a base 338 of the rotation mechanism. The rotation mechanism 330 may further include a stage 340 rotatably mounted to the bracket 336. According to various embodiments, the rotational axis of the stage 340 and the rotational axis of the bracket 336 may be orthogonal to each other. Further, the bioreactor chamber 310 may be coupled to the stage 340 such that the bioreactor chamber 310 may be rotated by the rotation of the stage 340 and the bracket 336 of the rotation mechanism 330.

According to various embodiments, the apparatus 300 may further include at least one other stimulator 350 coupled to the bioreactor chamber 310. The at least one other stimulator 350 may include a mechanical stimulator 351 configured to apply a mechanical stimulation to the seeded-scaffold 321 on the shaft 324 of the scaffold holder 320. As shown, the mechanical stimulator 350 may include a compression motor assembly 352 coupled to the lid 316 of the bioreactor chamber 310.

Accordingly, the compression motor assembly 352 may be in connection with the shaft 324 of the scaffold holder 320 such that the compression motor assembly 352 may be configured to move or slide the shaft 324 in a direction at least substantially perpendicular to the lid 316 for relatively moving the shaft 324 inward and outward with respect to the lid 316. Since the retainer 322 is at the free-end of the shaft 324, when the shaft 324 is being moved outward with respect to the lid 316 (i.e. analogous to the movement of pulling the shaft 324 out of the bioreactor chamber 310 from the lid 316), the seeded-scaffold 321 held on the shaft 324 may be sandwiched between the lid 316 and the retainer 322 such that the seeded-scaffold 321 may be compressed. On the other hand, when the shaft 324 is being moved inward with respect to the lid 316 (i.e. analogous to the movement of pushing the shaft 324 through the lid 316 into the bioreactor chamber), the compressive force on the seeded-scaffold 321 may be eased. Accordingly, cyclic inward and outward movement of the shaft 324 through the lid 316 of the bioreactor chamber 310 may cause cyclic compression on the seeded-scaffold 321.

FIG. 3C shows the bioreactor chamber assembly 311 including the bioreactor chamber 310 and the mechanical stimulator 351 coupled to the lid 316 of the bioreactor chamber 310. While the bioreactor chamber 310 may be spherical, the bioreactor chamber 310 may include a chamber base 313 configured for coupling with the stage 340 of the rotation mechanism 330. For example, the chamber base 313 may be shaped or profiled with a suitable depression or recess to receive the spherical bioreactor chamber 310, and also shaped or profiled with an interlocking element for engagement with a corresponding interlocking element on the stage 340 of the rotation mechanism 330.

FIG. 3D shows an exploded view of the bioreactor chamber assembly 311 of FIG. 3C according to various embodiments. As shown, the mechanical stimulator 351 may be coupled with the scaffold holder 320. In particular, the shaft 324 of the scaffold holder 320 may be coupled to the mechanical stimulator 351 such that the mechanical stimulator 351 may generate a motion to be transmitted to the shaft 324 for moving the shafts 324. For example, the mechanical stimulator 351 may generate a linear motion which may be transmitted to the shaft 324 for linearly moving the shaft 324 along respective longitudinal axis. Further, the shaft 324 of the scaffold holder 320 may be inserted perpendicularly through the lid 316 of the bioreactor chamber 310. Accordingly, when the shaft 324 of the scaffold holder 320 is moved by the mechanical stimulator 351, the shaft 324 of the scaffold holder 320 may be moved inward and outward with respect to the lid 316. As also shown, when the shaft 324 of the scaffold holder 320 is inserted through the lid 316, the seeded-scaffold 321 as well as spacers 326 may be placed onto the shaft 324 of the scaffold holder 320 such that the shaft 324 may pass through the hollow channel of the seeded-scaffold 321 and the spacers 326. Afterwhich, the retainer 322 may be coupled to the free-end of the shaft 324 of the scaffold holder 320. Furthermore, with the scaffold holder 320 having the seeded-scaffold 321 coupled to the lid 316, the lid 316 may be coupled to the main container body 314 of the bioreactor chamber 310 such that the scaffold holder 320 may be suspended inside the bioreactor chamber 310. According to various embodiments, the lid 316 may include fluid inlet and fluid outlet as well as sensors/wirings. The former configured for fluid connection with fluid medium/media reservoir such that the bioreactor chamber 310 may be filled with fluid medium/media and the latter configured for process monitoring and control.

FIG. 3E shows a cross-sectional view of the bioreactor chamber assembly 311 of FIG. 3C according to various embodiments. FIG. 3F shows an exploded view of the mechanical stimulator 351 of the bioreactor chamber assembly 311 of FIG. 3C according to various embodiments. As shown the mechanical stimulator 351 may include a motor 353 coupled to a drive shaft 354. A first spiral miter gear 355 may be fixedly coupled to the drive shaft 354 such that the spiral miter gear 355 is coaxial with the drive shaft 354. Accordingly, the motor 353 may drive a rotation of the first spiral miter gear 355 via the drive shaft 354. The mechanical stimulator 351 may further include an auxiliary shaft 356 arranged perpendicular to the drive shaft 354. A second spiral miter gear 357 may be fixedly coupled to an end of the auxiliary shaft 356 in a coaxial arrangement. Accordingly, the second spiral miter gear 357 may mesh with the first miter gear 355 such that rotating the first spiral miter gear 355 may drive a rotation of the second spiral miter gear 357 and the auxiliary shaft 356. Further, a first ground helical gear 358 may be fixedly coupled to the auxiliary shaft 356 in a coaxial arrangement. Accordingly, the first ground helical gear 358 may rotate together with the auxiliary shaft 356. The mechanical stimulator 351 may further include a compression shaft 359. The compression shaft 359 may be a rod with external screw thread. Further, a second ground helical gear 360 having internal screw thread may be screwed onto the compression shaft 359. The compression shaft 359 may be arranged to be parallel to the auxiliary shaft 356 and disposed such that the second ground helical gear 360 may mesh with the first ground helical gear 358. Accordingly, rotating the first ground helical gear 358 may rotate the second ground helical gear 360. Further, rotation of the second ground helical gear 360 would cause a linear motion of the compression shaft 359 along its longitudinal axis as a result of the internal screw thread of the second ground helical gear 360 engaging the external screw thread of the compression shaft 359. Accordingly, through the arrangement of the gears and shafts, the motor 353 may transmit a rotation which may be converted to the linear motion of the compression shaft 359 along its longitudinal axis. Hence, the shafts 324 of the scaffold holder 320 may be coupled to the compression shaft 359 such that linear motion of the compression shaft 359 may drive corresponding linear motion of the shafts 324 of the scaffold holder 320. According to various embodiments, the first spiral miter gear 355 and the second spiral miter gear 357 may be replaced with a pair of straight bevel gears or a pair of spiral bevel gears. According to various embodiments, the first ground helical gear 358 and the second ground helical gear 360 may be replaced with a pair of straight-cut gears, a pair of skew gears, or a pair of double helical gears. According to various embodiments, the mechanical stimulator 351 may include one or more bearings 361 coupled to the shafts 354, 356, 359 and/or the gears 355, 357, 358, 360 to facilitate rotations of the respective components. Further, the mechanical stimulator 351 may include one or more casing parts 362 configured to enclose or encase the arrangement of shafts 354, 356, 359 and the gears 355, 357, 358, 360.

Referring back to FIG. 3A and FIG. 3B, the apparatus 300 further include a perfusion flow system 371 as another form of stimulator. The perfusion flow system 371 may include the pump 380 in fluid connection with the bioreactor chamber 310. The perfusion flow system 371 may also include at least one medium reservoir 370 in fluid connection with the bioreactor chamber 310 and the pump 380. Hence, the pump 380, the at least one medium reservoir 370 and the bioreactor chamber 310 may be in fluid connection forming a loop such that the pump 380 may pump the medium from the at least one medium reservoir 370 into the bioreactor chamber 310 for perfusing through the seeded-scaffold in the bioreactor chamber 310 and back into the at least one medium reservoir 370. According to various embodiments, the medium may be perfused through the seeded-scaffold in the bioreactor chamber 310 by pumping the medium via the pump 380 based on a predetermined pump rate.

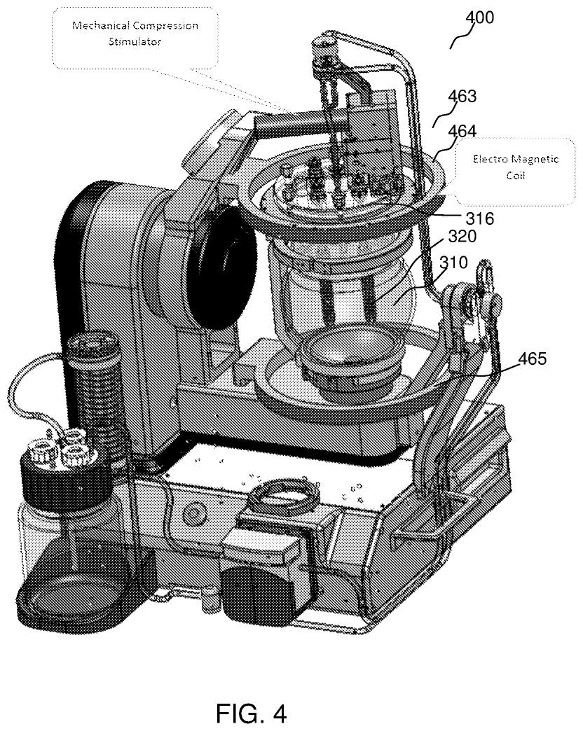

FIG. 4 shows an apparatus 400 for in-vitro tissue cultivation according to various embodiments. As shown, the apparatus 400 of FIG. 4 differs from the apparatus 300 of FIG. 3A to FIG. 3B in that the apparatus 400 of FIG. 4 may include an additional stimulator in the form of a magnetic stimulator 463. The magnetic stimulator 463 may include an electromagnet arrangement configured to apply a magnetic stimulation to the seeded-scaffold in the bioreactor chamber 310. As shown, the electromagnet arrangement may include one set of Helmholtz coil. The one set of Helmholtz coil may include two solenoid electromagnets 464, 465 arranged on a same axis. As shown, the two solenoid electromagnet 464, 465 may be coaxial with a vertical axis of the bioreactor chamber 310. Accordingly, one solenoid electromagnet 464 of the one set of Helmholtz coil may be disposed at the lid 316 of the bioreactor chamber 310 (or a top of the bioreactor chamber) and one other solenoid electromagnet 465 of the one set of Helmholtz coil may be disposed at a base of the bioreactor chamber 310 (or a bottom of the bioreactor chamber) such that the two solenoid electromagnets of the one set of Helmholtz coil may be in a coaxial arrangement. In this configuration, a magnetic field may be generated in a direction parallel to the longitudinal axis of the scaffold holder 320.

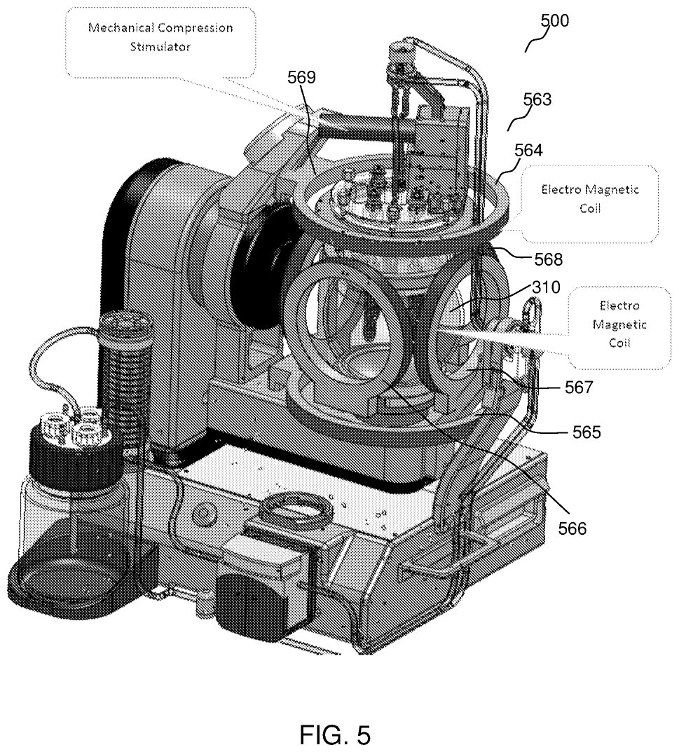

FIG. 5 shows an apparatus 500 for in-vitro tissue cultivation according to various embodiments. As shown, the apparatus 500 of FIG. 5 differs from the apparatus 400 of FIG. 4 in that the magnetic stimulator 563 of the apparatus 500 of FIG. 5 may include three sets of Helmholtz coils arranged along three orthogonal axes. Accordingly, six solenoid electromagnets 564, 565, 566, 567, 568, 569 may box up the bioreactor chamber 310 such that there is a top solenoid electromagnet 564, a bottom solenoid electromagnet 565 and four side solenoid electromagnets 566, 567, 568, 569 arranged in a box arrangement. The top solenoid electromagnet 564 and the bottom solenoid electromagnet 565 may be in a coaxial arrangement. A first pair of solenoid electromagnets 566, 568 of the four side solenoid electromagnets may be in a coaxial arrangement. Similarly, a second pair of solenoid electromagnets 567, 569 of the four side solenoid electromagnets may also be in a coaxial arrangement. Further, the axis of the first pair of solenoid electromagnets 566, 568 may be orthogonal to the axis of the second pair of solenoid electromagnets 567, 569. Accordingly, the four side solenoid electromagnets 566, 567, 568, 569 may form four side walls of a square. Therefore, with the six solenoid electromagnets arrangement (or the three sets of Helmholtz coils), a magnetic field through the bioreactor chamber 310 may be generated in any direction.

Various embodiments have provided a method and apparatus (including a bioreactor chamber or a cell culture chamber) for growing three dimensional cell cultures in a dynamic environment with mechanotransduction capabilities. Various embodiments may be applied in research activities involving tissue engineering and regenerative medicine and disease modeling so as to reduce the use of animal models, as well as future healthcare industry applications.

Various embodiments have provided a physiological bioreactor chamber which may function as part of a multimodal bioreactor system that may allow the application of cyclic compressive strains on premature bone grafts that are cultured under biaxial rotation (chamber rotation about two axes) conditions for bone tissue engineering. Various embodiments may also be integrated with sensors for dissolved oxygen levels and pH (not limited to these only) that allow real-time, non-invasive monitoring of the culture parameters. The apparatus (or the bioreactor system) may be capable of four different modes of operations: namely static mode, cyclic compression mode, biaxial rotation mode and multimodal mode (i.e. combination of cyclic compression and biaxial rotation). The applications of the methods and apparatus of the various embodiments are also not limited to bone but also to other tissue types.

Various embodiments may enable the regenerative tissue to grow in all directions and not be dictated by a single signaling vector. In various embodiments, the bioreactor chamber (or the physiological chamber) may be mounted onto the biaxial bioreactor system, so that it may be possible to achieve not only optimal cell culture conditions but also promote the benefits of enhanced osteogenesis in in-vitro cultured grafts.

Various embodiments have provided a physiological bioreactor chamber and a method for growing three dimensional cell cultures that achieve physical signaling in more than one force vector or flow vector or both. Various embodiments have also provided a system, an apparatus and a method for growing three dimensional cell or tissue cultures in-vitro.

According to various embodiments, the bioreactor chamber (or the physiological bioreactor chamber) may be of either a spherical-shaped chamber or other shaped culture chamber for growing three dimensional cell or tissue cultures. Accordingly, the bioreactor chamber (or the physiological bioreactor chamber) may include a spherical-shaped or other shaped culture chamber for containing a cell or tissue culture, a culture medium, scaffold holder/s, physiological actuator/s for growing cell or tissue cultures. The bioreactor chamber may sit on a slider-lock that engages the chamber to the biaxial bioreactor system. Customized electrical interfaces may connect the chamber electrical/actuator/sensing system to the controllers for process controls and monitoring requirements (whilst achieving simultaneous biaxial chamber rotations).

According to various embodiments, the flow regime generated within the spherical culture chamber, by the biaxial rotary system, may create a non-turbulent, low vortex flow that reduces cell damage and optimizes the expression of differentiated function and enhancing osteogenesis and tissue development.

According to various embodiments, the multimodal physiological bioreactor chamber may allow the application of cyclic compressive strains on grafts that may be cultured under biaxial rotation (chamber rotation about two axes) conditions for tissue engineering. The physiological chamber/bioreactor may be integrated with sensors for dissolved oxygen levels and pH that allow real-time, non-invasive monitoring of the culture parameters. The grafts cultured in this system may be subjected to force or flow vectors that are associated with four different modes of operation—static, cyclic compression (or cyclic tension, or cyclic shear, or cyclic torsion, or cyclic bending), biaxial rotation and multimodal. The multimodal mode may for example include a combination of the cyclic compression (or cyclic tension, or cyclic shear, or cyclic torsion, or cyclic bending) and the biaxial rotation. These modes of operations may allow fluid shear stresses and mechanical strains to stimulate the cell membrane's structural proteins to form focal adhesion complexes which in turn may enable transduction of these specific mechanical signals to respective biochemical signals. Adequate mass transport of chemical species i.e. CO2, oxygen etc. within the chamber may also be achieved dynamically by the bi-axially rotating drive system that rotates the bioreactor chamber about two axes (x-axis and z-axis) simultaneously whilst maintaining physiological stresses on the culturing grafts. The multimodal physiological bioreactor chamber may include a media reservoir (500 mL, 1 L or other volumetric sizes), gas-permeable tubing and an inlet/an outlet port for media perfusion and one or more mechanical micromanipulators to apply cyclic compressive force on scaffolds. The chamber lid may include two or more shafts (or scaffold holder) to anchor the scaffolds and each shaft can accommodate several scaffolds. A motor may provide the rotary torque to a first gear via a gear train arrangement and the rotational torque may be converted to linear shaft motion through a series of gears to achieve precise displacement capabilities. The vertical translation may cause scaffolds loaded onto the shaft (or scaffold holder) to be subjected to pre-determined compressive forces (tension, shear, bending and torsion forces acting individually or in synchronization is/are also possible with the use of additional accessories). The parameters for media perfusion, biaxial rotation and physiological loading may be controlled by an external controller unit. Oxygen spot sensors may be mounted to the inner wall of the chamber for real-time process monitoring. The bioreactor chamber may be steam sterilisable along with all the components that come into contact with the media. The modes of operations may include ‘Static mode’ which allows only media perfusion using the variable speed peristaltic pump. ‘Cyclic compression mode’ of operation may provide scaffold compression of predetermined percentage and at a predetermined frequency. ‘Biaxial rotation mode’ of operation may provide chamber rotation about two axes (x-axis and z-axis) simultaneously. ‘Multimodal mode’ of operation may be a combination of biaxial rotation and at least one other stimulation, such as a mechanical stimulation (for example, cyclic compression, cyclic torsion, cyclic shear, cyclic tension or cyclic bending), a magnetic stimulation (for example using Helmholtz coil(s)), an electrical stimulation, a stimulation with one or more light sources, or any other suitable stimulation or combination of stimulations at predetermined parameters. Non-invasive sensing features may also be provided for dissolved oxygen, pH and/or other probes necessary for process monitoring.

According to various embodiments, the apparatus including the bioreactor chamber with one or more stimulator and the biaxial rotation mechanism (i.e. the multimodal physiological bioreactor chamber used in conjunction with the biaxial bioreactor system) may enable cell cultures to attain higher and rapid cellular proliferation with improved matrix deposition. The combined effects of optimal fluid flow conditions and cyclic compression may lead to osteogenic grafts marked by elevated expressions of osteogenic genes after 2 week culture. The results confirmed that the combination of cyclic mechanical stimulation and biaxial rotation may promote or enhance maturation of cellular bone grafts.

In the following, experiments and experimental results conducted based on a method and an apparatus according to the various embodiments are provided as an example illustration of the performance and technical effects of the various embodiments.

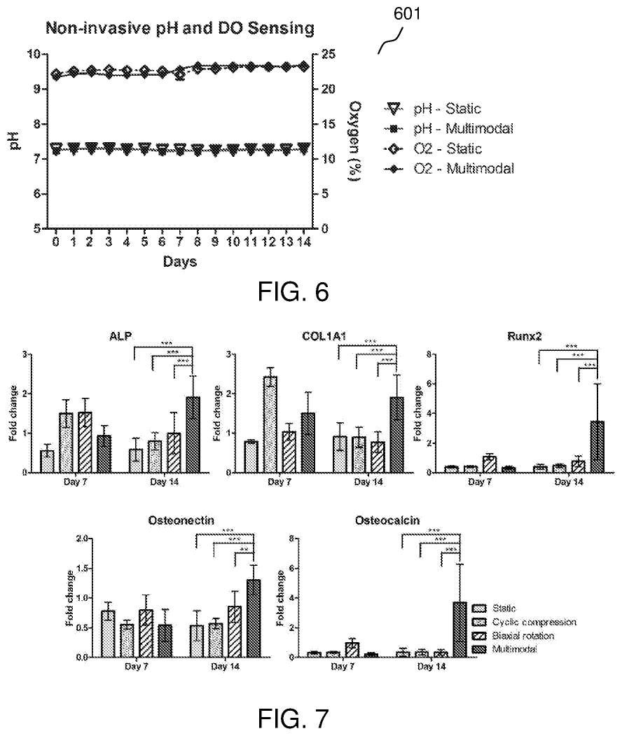

According to the experiments conducted, the physiologic bioreactor runs were conducted under four different modes. Static mode is maintained in the bioreactor chamber with only media perfusion using the peristaltic pump (Speed: 5 rpm). This would act as the baseline for all the modes. Cyclic compression mode would include compression at 0.22%, 1 Hz for 4 hours/day. Biaxial rotation mode would involve chamber rotation about two axes (x-axis and z-axis) at 5 rpm and an angle of rotation at 90°. The final mode would be the multimodal mode which is a combination of biaxial rotation and cyclic compression at the same parameters. The dissolved oxygen levels (%) in the chamber media were read out non-invasively from four sensor spots using transmitters by halting the system twice a day. The pH of the media was detected from the flow-through pH sensor twice a day in a non-invasive manner. The culture parameters (pH and dissolved oxygen) were determined online in a non-invasive manner over the 2 weeks of bioreactor culture. The preliminary results indicated that the pH was maintained at an average of 7.2 over the entire culture period. The dissolved oxygen was also maintained throughout the culture period under both static and bioreactor conditions. FIG. 6 shows a plot 601 depicting the maintenance of pH (left y-axis) and dissolved oxygen (02) levels (right y-axis) over two weeks under static mode and multimodal mode bioreactor conditions measured using non-invasive sensing modalities. As shown, there are no observable differences in terms of the dissolved oxygen and pH levels between the different modes/groups. This suggests that any observed effects on cellular proliferation and differentiation between the different modes/groups in the experiments were solely due to the fluid dynamics and/or mechanical stimulation in the respective modes/groups.

FIG. 7 shows the expression levels of osteogenic genes—ALPL (alkaline phosphatase, tissue-nonspecific isozyme), COL1A1 (collagen, type I, apha 1), Runx2 (runt-related transcription factor 2), Osteonectin and Osteocalcin under the different modes of bioreactor assessed using Reverse Transcription Polymerase Chain Reaction (RT-PCR) with respect to day 0 undifferentiated Mesenchymal Stem Cells (MSC) controls (n=3, ** p<0.01, *** p<0.001). The primer sequences for genes used in RT-PCR are shown in Table 1 below. Differential expression of osteogenic genes under different culture conditions was evaluated using RT-PCR and the fold change for all the modes/groups is reported with respect to undifferentiated MSC controls as shown in FIG. 7. In the first week of bioreactor culture, expression fold change of COL1A1 was higher or upregulated in the groups experiencing cyclic compressive stimuli (cyclic compression mode: 3×, p<0.001; multimodal mode: 1.9×, p<0.01) in comparison to static cultures. Expression fold change of ALP was found to be higher (2.7×) or upregulated in individual stimulation modes (biaxial rotation mode and cyclic compression mode) in comparison to the static mode. By the end of second week combination of biaxial rotation and cyclic compression in the multimodal mode led to upregulation in the expression of osteogenic genes including ALP (3.2×, p<0.001), COL1A1 (2×, p<0.001), Runx2 (8.6×, p<0.001), osteonectin (2.4×, p<0.001) and osteocalcin (10×, p<0.001) in comparison to static cultures.

TABLE 1
Primer sequences for genes used in RT-PCR

| | | PrimerBank ID/ | Sequence |
|---|---|---|---|
| Gene | Primer sequences | Reference | number |
| ALPL | F: ACTGGTACTCAGACAACGAGAT | 294660769c2 | Sequence 1 |
| | R: ACGTCAATGTCCCTGATGTTATG | | Sequence 2 |
| Osteonectin | F: AGCACCCCATTGACGGGTA | 4507171a1 | Sequence 3 |
| | R: GGTCACAGGTCTCGAAAAAGC | | Sequence 4 |
| Osteocalcin | F: CACTCCTCGCCCTATTGGC | 4502401a3 | Sequence 5 |
| | R: GCCTGGGTCTCTTCACTACCT | | Sequence 6 |
| COL1A1 | F: AGGACAAGAGGCATGTCTGGTT | Zhang Z-Y, Teoh SH, | Sequence 7 |
| | R: CCCTGGCCGCCATACTC | Chong W-S, et al. | Sequence 8 |
| | | 2009a, A biaxial | |
| | | rotating bioreactor for | |
| | | the culture of fetal | |
| | | mesenchymal stem | |
| | | cells for bone tissue | |
| | | engineering, | |
| | | Biomaterials, 30 | |
| | | (14): 2694-2704. | |
| Runx2 | F: AGTAGGTGTCCCGCCTCAGA | Zhang ZY, Teoh SH, | Sequence 9 |
| | R: CCHGTGGAHAAAAGGACTTGGT | Chong MS, et al. | Sequence 10 |
| | | 2009b, Superior | |
| | | osteogenic capacity | |
| | | for bone tissue | |
| | | engineering of fetal | |
| | | compared with | |
| | | perinatal and adult | |
| | | mesenchymal stem | |
| | | cells, Stem Cells, 27 | |
| | | (1): 126-137. | |

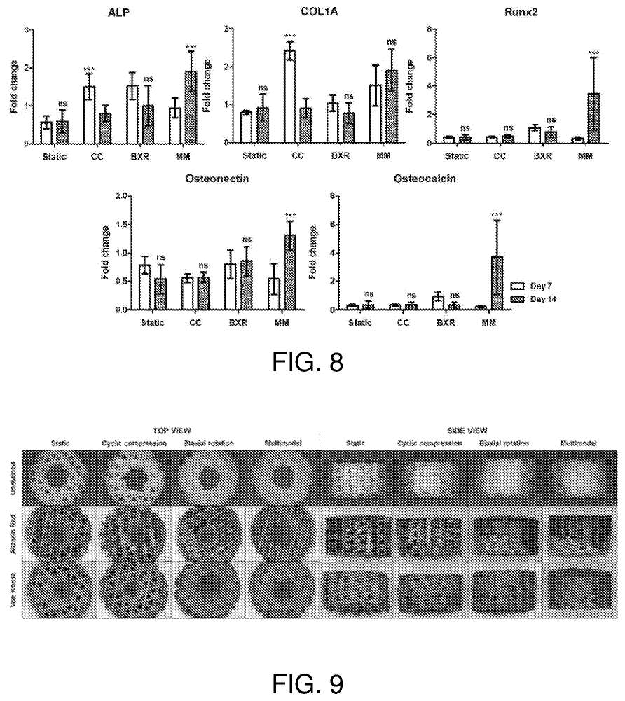

FIG. 8 shows fold increment and statistical significance in each bioreactor condition from 7 to 14 days. FIG. 8 shows Osteogenic gene expression on day 7 vs day 14 under different modes of bioreactor—Static mode, CC (Cyclic compression mode), BXR (Biaxial rotation mode), MM (Multimodal mode)) assessed using RT-PCR with respect to day 0 undifferentiated MSC controls. FIG. 8 also show statistical significance in each bioreactor condition day 7 vs day 14 (ns: p>0.05, *** p<0.001). As shown, selected fold increments in gene expression in each condition with respect to day 7 are as follows: ALP (MM: 2×), COL1A (CC: 0.37×), Runx2 (MM: 10×), Osteonectin (2.4×), Osteocalcin (16×).

With respect to osteogenic differentiation, combination of biaxial rotation and cyclic compression in the multimodal mode showed significant upregulation of osteogenic gene expression by the end of second week of bioreactor culture as can be seen from FIG. 7. Runx2 is a master regulator gene that is expressed early during osteoblastogenesis. According to the experimental results, Runx2 was found to be highly upregulated in the multimodal mode in comparison to the other modes by the end of 2 weeks of culture. COL1A1 is another important marker of osteogenic differentiation of MSCs. According to the experimental results, higher expressions of COL1A1 gene were found in the cyclic compression groups (cyclic compression mode and multimodal mode) in the first week of culture in comparison to the rest of the modes. These results indicate potential role played by physiologically relevant mechanical stimuli in the induction of cellular differentiation in a multimodal configuration. Further, the upregulated expression of COL1A1 was sustained till the end of the 2 weeks of culture in multimodal conditions unlike the cyclic compression mode where the expression levels dropped after day 7 (as can be seen from FIG. 7 and FIG. 8).

Further, from FIG. 8, it can be seen that in static mode, cyclic compression mode and biaxial rotation mode, the expression levels of the respective genes between day 7 and day 14 are either about the same or have dropped. In contrast, in multimodal mode, the upregulated expressions of the respective genes were sustained. Accordingly, the synergistic effects of cyclic compression and fluid shear stresses in the multimodal mode bioreactor platform are clearly reflected in the upregulated osteogenic gene expressions. Accordingly, the combined effects in the multimodal mode platform according to the various embodiments have proven to be beneficial for increased osteogenic differentiation of stem cells on 3D scaffolds. Further, the time-dependent differential gene expression from the experimental results may also suggest the importance of critical role played by timing of application of each stimulus in a multimodal configuration.

FIG. 9 shows qualitative staining for calcium and phosphate in the tissues by the end of week 2. Alizarin red staining for calcium shows the distribution of calcium deposits on the tissue as does the Von Kossa staining for the phosphates. Results showed that there were visible differences in the levels of calcium and phosphate deposition in the first 2 weeks of culture under the different modes of bioreactor culture. The biaxial rotation mode/group and multimodal mode/group showed higher deposition and matrix filled pores in comparison to the poorly cellularized grafts with empty pores in the static and cyclic compression modes/groups. The side view of the grafts indicate a non-homogeneous distribution with more number of cells on just the top region (cell seeding side) under all the modes of bioreactor culture. The biaxial rotation and multimodal modes/groups exhibited higher deposition of mineralized matrix in comparison to the static and cyclic compression modes/groups.

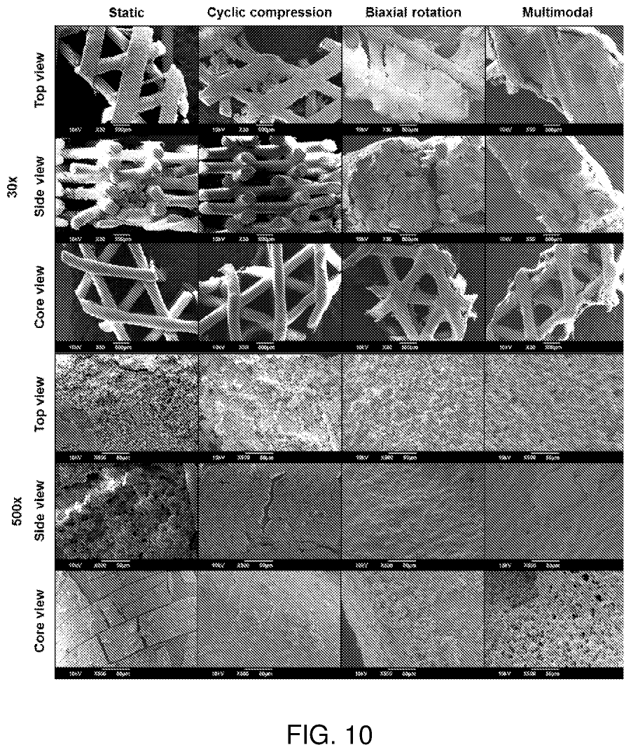

FIG. 10 shows matrix deposition in the tissue grafts under different bioreactor modes, SEM images of the top view, side view and core views (30×, 500×) show matrix deposition on the scaffolds and the pores of the scaffolds under all four modes of bioreactor culture at the end of week 2. As shown, the scaffolds under biaxial rotation and multimodal cultures showed enhanced deposition of matrix on the scaffold struts and the pores (top view and side view). In the core of the scaffolds, little or no deposition of matrix was observed when they were cultured under static or cyclic compression modes. On the other hand, the scaffolds cultured under biaxial rotation and multimodal modes showed moderate matrix deposition.

As illustrated by the experiment above, various embodiments have allowed the combination of perfusion, cyclic compression and biaxial rotation for engineering bone tissues. Further, the results have demonstrated the potential advantages of the various embodiments in integrating non-invasive sensing modalities with bioreactor culture to sense culture parameters. As illustrated, various embodiments have also provided a platform that allows real-time sensing of the oxygen levels for future studies that can analyze the effects of the hypoxic conditions combined with dynamic bioreactor conditions for osteogenic differentiation.

As illustrated, a platform according to the various embodiments has been developed and evaluated to engineer bone tissues using the physiologic bioreactor system that allows application of relevant compressive forces and an advantageous fluid flow regime. The advantages of using the physiologic bioreactor for generation and maintenance of bone grafts under physiologically relevant conditions have been demonstrated from the results discussed above. As shown, multimodal culture in the physiologic bioreactor resulted in higher and rapid cellular proliferation and improved matrix deposition. The combined effects of optimal fluid flow conditions and cyclic compression led to osteogenic grafts marked by elevated expressions of osteogenic genes after 2-week culture. The results confirmed that the combination of cyclic mechanical stimulation and biaxial rotation will promote maturation of cellular bone graft. The results have demonstrated the potential advantages of using the apparatus according to the various embodiments (i.e. the multimodal system) for generation and maintenance of bone grafts culture under physiologically relevant conditions. Further, various embodiments have also provided an apparatus for in-vitro tissue cultivation which may be configured to enable effective translation for future clinical utility for generation of autologous bone grafts using patient's own cells.

While the invention has been particularly shown and described with reference to specific embodiments, it should be understood by those skilled in the art that various changes, modification, variation in form and detail may be made therein without departing from the scope of the invention as defined by the appended claims. The scope of the invention is thus indicated by the appended claims and all changes which come within the meaning and range of equivalency of the claims are therefore intended to be embraced.

1. A method for in-vitro tissue cultivation, the method comprising:
providing a seeded-scaffold to a scaffold holder suspended in a medium contained in a bioreactor chamber;
rotating, via a rotation mechanism which the bioreactor chamber is coupled to, the bioreactor chamber about two orthogonal axes based on a predetermined motion cycle as a stimulation for tissue growth; and
applying, via a stimulator coupled to the bioreactor chamber, at least one other stimulation for tissue growth to the seeded-scaffold.

2. The method as claimed in claim 1, wherein the scaffold holder is elongated, and wherein providing the seeded-scaffold comprises placing the seeded-scaffold to surround the scaffold holder and retaining the seeded-scaffold on the scaffold holder with a retainer, and wherein the scaffold holder extends from a portion of a ceiling of the bioreactor chamber to a center region of the bioreactor chamber.

3. (canceled)

4. The method as claimed in claim 1, wherein rotating the bioreactor chamber via the rotation mechanism comprises
rotating a bracket rotatably mounted to a base of the rotation mechanism, and
rotating a stage rotatably mounted to the bracket,
wherein a rotational axis of the stage is orthogonal to a rotational axis of the bracket, and
wherein the bioreactor chamber is coupled to the stage.

5. The method as claimed in claim 1, wherein the predetermined motion cycle is defined by one or more parameters selected from the group consisting of an angle of rotation, a direction of rotation, a rotational speed, a sequence of rotation, a period of rotation and a time interval between rotations.

6. The method as claimed in claim 1, wherein applying the at least one other stimulation for tissue growth to the seeded-scaffold comprises applying a mechanical stimulation to the seeded-scaffold, and wherein applying the mechanical stimulation to the seeded-scaffold comprises moving the scaffold holder, via an actuator of the stimulator, relative to the bioreactor chamber to apply a compression or a tension to the seeded-scaffold based on a predetermined mechanical stimulation cycle.

7. (canceled)

8. The method as claimed in claim 6, wherein the predetermined mechanical stimulation cycle is defined by one or more parameters selected from the group consisting of an amount of relative movement, a speed of relative movement, a period of relative movement, a sequence of relative movement, a force applied, and a time interval between compressions or tensions.

9. The method as claimed in claim 1, wherein applying the at least one other stimulation for tissue growth to the seeded-scaffold comprises applying a magnetic stimulation to the seeded-scaffold, and wherein applying the magnetic stimulation to the seeded-scaffold comprises generating a magnetic field, via an electromagnet arrangement of the stimulator, through the bioreactor chamber based on a predetermined magnetic stimulation cycle.

10. (canceled)

11. The method as claimed in claim 9, wherein the predetermined magnetic stimulation cycle is defined by one or more parameters selected from the group consisting of a magnitude of the magnetic field, a magnetic flux, a direction of the magnetic field, a period of magnetic field generation, a sequence of magnetic field generation, and a time interval between magnetic field generations.

12. The method as claimed in claim 9, wherein the electromagnet arrangement comprises a set of Helmholtz coils or three sets of Helmholtz coils arranged along three orthogonal axes.

13. The method as claimed in claim 1, wherein applying the at least one other stimulation for tissue growth to the seeded-scaffold comprises perfusing the medium through the seeded-scaffold in the bioreactor chamber by pumping the medium, via a pump in fluid connection with the bioreactor chamber and at least one medium reservoir, based on a predetermined pump rate.

14. An apparatus for in-vitro tissue cultivation, the apparatus comprising
a bioreactor chamber configured to contain a medium;
a scaffold holder which is suspended in the bioreactor chamber and which is configured to receive a seeded-scaffold;
a rotation mechanism as a stimulator for tissue growth, wherein the bioreactor chamber is coupled to the rotation mechanism, and wherein the rotation mechanism is configured to rotate the bioreactor chamber about two orthogonal axes based on a predetermined motion cycle; and
at least one other stimulator for tissue growth, wherein the at least one other stimulator is configured to apply at least one other stimulation to the seeded-scaffold, and wherein the at least one other stimulator is coupled to the bioreactor chamber.

15. The apparatus as claimed in claim 14, wherein the scaffold holder is elongated, wherein the scaffold holder is configured to receive the seeded-scaffold such that the seeded-scaffold surrounds the scaffold holder, and wherein a retainer retains the seeded-scaffold on the scaffold holder, and wherein the scaffold holder extends from a portion of a ceiling of the bioreactor chamber to a center region of the bioreactor chamber.

16. (canceled)

17. The apparatus as claimed in claim 14, wherein the rotation mechanism comprises
a bracket rotatably mounted to a base of the rotation mechanism, and
a stage rotatably mounted to the bracket,
wherein a rotational axis of the stage is orthogonal to a rotational axis of the bracket, and
wherein the bioreactor chamber is coupled to the stage.

18. The apparatus as claimed in claim 14, wherein the rotation mechanism is configured to define the predetermined motion cycle by one or more parameters selected from the group consisting of an angle of rotation, a direction of rotation, a rotational speed, a sequence of rotation, a period of rotation and a time interval between rotations.

19. The apparatus as claimed in claim 14, wherein the at least one other stimulator comprises a mechanical stimulator configured to apply a mechanical stimulation to the seeded-scaffold, and wherein the mechanical stimulator comprises an actuator configured move the scaffold holder relative to the bioreactor chamber to apply a compression or a tension to the seeded-scaffold based on a predetermined mechanical stimulation cycle for applying the mechanical stimulation to the seeded-scaffold.

20. (canceled)

21. The apparatus as claimed in claim 19, wherein the mechanical stimulator is configured to define the predetermined mechanical stimulation cycle by one or more parameters selected from the group consisting of an amount of relative movement, a speed of relative movement, a period of relative movement, a sequence of relative movement, a force applied, and a time interval between compressions or tensions.

22. The apparatus as claimed in claim 14, wherein the at least one other stimulator comprises a magnetic stimulator configured to apply a magnetic stimulation to the seeded-scaffold, and wherein the magnetic stimulator the bioreactor chamber based on a predetermined magnetic stimulation cycle for applying the magnetic stimulation to the seeded-scaffold.

23. (canceled)

24. The apparatus as claimed in claim 22, wherein the magnetic stimulator is configured to define the predetermined magnetic stimulation cycle by one or more parameters selected from the group consisting of a magnitude of the magnetic field, a magnetic flux, a direction of the magnetic field, a period of magnetic field generation, a sequence of magnetic field generation, and a time interval between magnetic field generations.

25. The apparatus as claimed in claim 23, wherein the electromagnet arrangement comprises a set of Helmholtz coil or three sets of Helmholtz coils arranged along three orthogonal axes.

26. The apparatus as claimed in claim 14, wherein the at least one other stimulator comprises perfusion flow system, wherein the perfusion flow system comprises a pump in fluid connection with the bioreactor chamber and at least one medium reservoir, and wherein the pump is configured to pump the medium through the bioreactor chamber based on a predetermined pump rate to perfuse the medium through the seeded-scaffold.
